(19) United States
(12) Patent Application Publication
(10) Pub. No.: US 2003/0219850 A1
(43) Pub. Date: Nov. 27, 2003

(54) Method for automatically analyzing nucleated bone marrow cells

(75) Inventors: Tomohiro Tsuji, Kobe-shi (JP); Yuji Itose, Kobe-shi (JP); Aya Konishi, Kobe-shi (JP)

Correspondence Address: Lance J. Lieberman, Esq., Cohen, Pontani, Lieberman & Pavane, Suite 1210, 551 Fifth Avenue, New York, NY, 10176, US

(73) Assignee: Sysmex Corporation

(21) Appl. No.: 10/436,865

(22) Filed: May 13, 2003

(30) Foreign Application Priority Data
May 16, 2002 (JP) 2002-141958

(51) Int. Cl. G01N 33/48

(52) U.S. Cl. 435/40.5

(57) ABSTRACT

An automatic method for analyzing nucleated bone marrow cells comprising partitioning one sample of bone marrow fluid with two samples, one sample being treated with a first lysing agent and a first staining solution and the other sample being treated with a second lysing agent and a second staining solution; and

measuring each samples in a flow cytometer using a scattered light and fluoresence which enables to classify and count leukocytes, erythroid cells and lipid particles, as well as mature myeloid cells, lymphoid cells and immature myeloid cells, and to calculate the number of myeloid cells as well as the ratio of myeloid cells and erythroid cells.

### CROSS-REFERENCE TO RELATED APPLICATION

This application is related to Japanese Patent Application No. 2002-141958 filed on May 16, 2002, whose priority is claimed under 35 USC §119, the disclosure of which is incorporated by reference in its entirety.

### BACKGROUND OF THE INVENTION

1. Field of the Invention

The present invention relates to a method for automatically analyzing nucleated bone marrow cells. More particularly, it relates to a method for analyzing nucleated cells of bone marrow fluid by means of flow cytometry.

2. Description of Related Art

In the field of laboratory tests, the analyzing of nucleated bone marrow cells helps to obtain extremely useful information for diagnosing diseases. For example, normal bone marrow commonly contains specific proportions of nucleated bone marrow cells such as myeloid cells, erythroid cells and others. Some diseases cause changes in the number of nucleated cells, e.g., erythroid cells, myeloid cells and others, which sometimes results in changes in the proportions of myeloid cells and erythroid cells (Myeloid/Erythroid ratio, hereinafter referred to as M/E ratio).

For example, as diseases increasing the number of nucleated bone marrow cells, acute leukemia of various types, myelodysplastic syndrome (MDS), chronic myelogenous leukemia (CML) and others are mentioned. As diseases decreasing the number of nucleated bone marrow cells, anaplastic anemia and hypoplastic leukemia and others are mentioned. Also, as diseases decreasing the number of erythroid cells, erythrocytic phthisis and others are mentioned. As diseases increasing the number of myeloid cells, leukemia of various types and malignant lymphoma and others are mentioned. Furthermore, in the case of anemia, erythroid cells increase. Also, as diseases increasing M/E ratio, leukemia of various types and malignant lymphoma and others are mentioned. As diseases decreasing M/E ratio, agranulocytosis and anemia of various types are mentioned. Especially, acute leukemia is divided according to FAB classification with use of nucleated bone marrow cell count and M/E ratio. Thus, it is very useful for diagnosing diseases and measuring hematopoiesis to classify and count nucleated bone marrow cells, myeloid cells and erythroid cells and to obtain M/E ratio.

Various kinds of components contained in bone marrow have usefully been analyzed by producing a smear sample of bone marrow, staining the sample with suitable dye and microscopically observing the stained sample.

In recent years, various kinds of whole automated apparatuses for analyzing blood apparatuses, which utilize the principle of flow cytometry, are available. For example, a method for analyzing lymphoid cells, mature myeloid cells and immature myeloid cells is disclosed in U.S. Pat. No. 5,958,776: This method utilizing fluorescent dyes selectively staining the damaged mature myeloid cells except immature myeloid cells after other cells were damaged.

In this method, myeloid cells can be classified and counted, but erythroid cells cannot be analyzed. So, in another method, erythroid cells must be measured, and from the results obtained, M/E ratio was necessary to calculate. A simple and highly precised method was therefore expected to calculate M/E ratio.

### SUMMARY OF THE INVENTION

The present invention provides a method for automatic analysis of nucleated bone marrow cells which is possible to calculate M/E ratio at a simple and highly precised technique: The identical bone marrow fluid sample is, at first, divided into two samples, and each sample is hemolyzed and stained. Thus, erythorid cells and myeloid cells are measured at the same time.

More particularly it relates to a method for automatically analyzing nucleated bone marrow cells comprising the steps:

(1) preparing a first and second samples from a sample of bone marrow fluid;

(2) (i) preparing a first measuring sample obtained by mixing the first sample with a first lysing agent to lyse erythrocytes in the first sample, and mixing the mixture with a first staining solution containing at least a first fluorescent dye for producing a difference in each intensity of fluorescence among at least leukocytes, erythroid cells and lipid particles;

(ii) preparing a second measuring sample obtained by mixing the second sample with a second lysing agent to damage all the cells but immature myeloid cells, and mixing the mixture with a second staining solution containing at least a second fluorescent dye for producing a difference in each intensity of fluorescence between a group of mature myeloid cells and lymphoid cells and a group of immature myeloid cells;

(3) introducing each of the first and second measuring samples to a flow cytometer to measure at least one kind of scattered light and at least one kind of fluorescence;

(4) (i) classifying and counting leukocytes, erythroid cells and lipid particles with use of a difference in each intensity of the scattered light and the fluorescence of the first measuring sample;

(ii) classifying and counting mature myeloid cells, lymphoid cells and immature myeloid cells with use of a difference in each intensity of the scattered light and fluorescence of the second measuring sample;

(iii) calculating a number of myeloid cells from the counted mature myeloid cells and immature myeloid cells;

(5) calculating a ratio of myeloid cells and erythroid cells (M/E ratio) from the counted erythroid cells and myeloid cells.

Further, it provides an automatic analyzer for nucleated cells of bone marrow fluid.

### BRIEF DESCRIPTION OF THE DRAWINGS

FIG. 1 schematically shows the distribution of each components classified and counted in the scattergaram prepared with use of a difference in each intensity of the scattered light and fluorescence measured in step (3) on a sample stained in step (2)(i) of the present invention.

FIG. 2 schematically shows the population of components classified and counted in the scattergram produced with a difference in each intensity of (red) fluorescence and each intensity of scattered light in the sample measured in step (3) on a sample stained in step (2)(i) of the present invention.

FIG. 3 is a scattergram showing the population of neucleated bone marrow cells classified and counted by an embodiment of the present invention.

FIG. 4 is a scattergram showing the population of neucleated bone marrow cells classified and counted by the embodiment.

FIG. 5 schematically shows one whole system of an automatic analyzer of the present invention.

### DESCRIPTION OF THE PREFERRED EMBODIMENT(S)

The sample of bone marrow fluid in the present invention is that including a bone marrow aspirate which contains myeloid cells and erythroid cells and the like. In the present invention, the sample of bone marrow fluid does not particularly need pretreatment. However, if the presence of bone fragments or blood cell aggregates and the like disturbs the measurement of myeloid cells and erythroid cells, a filtration may be carried out as required. The sample of bone marrow fluid may be diluted with an aqueous solution containing a buffer, a chelating agent and/or an anticoagulant and the like. As the buffer, buffers as mentioned below may be used. As the chelating agent, EDTA salts and others may be used. The anticoagulant is not particularly limited, but heparin, citric acid or citrates, et al may be used. The sample may be diluted with the aqueous solution at dilution ratio suitably about 5- to 100-folds (by volume), preferably about 10- to 50-folds.

In the present invention, the sample of bone marrow fluid is divided into two samples in step (1). Each sample is introduced into a reaction chamber, which is individual or identical. The sample in the chamber is individually treated to obtain erythroid cell count and myeloid cell count from the same sample.

In the present invention, each of the sample is subjected to lysing and staining treatments in step (2).

In step (2)(i), the sample of bone marrow fluid is mixed with the first lysing agent. Thereby, erythrocytes contained in the sample of bone marrow fluid are lysed to such extent that they will not hinder the measurement of various kinds of cell components described later, and also leukocytes, erythroid cells and/or lipid particles are rendered condition suitable for staining. “The condition suitable for staining” means to be able to give any injury on cell membranes, but not to be damaged so as to maintain the function and form of the cells substantially as the living cell. As in erythrocytes, erythroid cells are pored in their cell membranes and lysed, but the condition of the cell nuclei of erythroid cells is substantially maintained as the condition of their living cells. It is not clear for what damage is given on the cell membranes of leukocytes, but leukocytes can be maintained substantially as the living cells, while any remarkable difference from the living cells are not observed by optical microscope.

The first lysing agent is not particularly limited so long as it exhibits such action. At osmotic pressure of 150 m Osm/kg or less, erythocytes typically are pored in their cell membranes, from which intracellular hemoglobin flows out, and erythocytes become optically transparent (are lysed), though some difference is seen between individual cells. The optically transparent erythrocytes do not disturb the measurement of various kinds of the cell components described later. Erythrocytes are lysed more rapidly at a lower osmotic pressure and a lower pH. Accordingly, the first lysing agent in the present invention preferably has an osmotic pressure of 100 m Osm/kg or less, more preferably about 30 to 100 m Osm/kg, in consideration of difference between sources of bone marrow fluids. If the pH is too low, the first lysing agent gives excessive damage not only to erythrocytes but also to leukocytes and erythorid cells, and so makes difficult to obtain the below-described difference in each intensity of fluorescence thereof. The lysing agent suitably has, therefore, an acid pH, preferably about 2.0 to 5.0, more preferably about 2.5 to 4.5.

In order to realize such an osmotic pressure and pH, the first lysing agent is preferably an aqueous solution containing an electrolyte, saccharide, buffer, etc. Furthermore, the first lysing agent is preferred to contain an organic acid and its salt having at least an aromatic ring in the molecule since erythrocytes can be lysed more effectively (more rapidly). Also, the first lysing agent preferably contains a surfactant.

As the electrolytes, NaCl, KCl and the like may be mentioned. As the saccharides, monosaccharides, polysaccharides, oligosaccharides such as glucose, lactose and sucrose may be mentioned. As the buffers, those having a pKa near pH ±2.0 to be set, and particular examples thereof include citric acid, malic acid, maleic acid, diglycolic acid, malonic acid or the like and salts thereof. As the organic acids and their salts, salicylic acid, phthalic acid and the like, their alkali metal salts (e.g., sodium salts, potassium salts, etc.) and the like may be mentioned. These also act as buffers.

The concentration of such components in the first lysing agent may be adjusted to about 0.1 to 100 mM, preferably about 1 to 30 mM.

In the first lysing agent, a surfactant may be any one so long as it can solubilize a slightly soluble dye, prevent erythrocyte ghosts from aggregation, prevent platelet from aggregation, shrink erythrocyte ghosts and/or promote erythrocyte lysis. For example, it is preferable to use the following surfactant singly or in combination of two or more, thereof. a compound of the formula (VI):

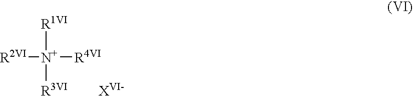

wherein, R^{1VI}, R^{2VI} and R^{3VI} respectively represent a hydrogen atom, a C₁₋₈ alkyl group or a C₆₋₈ aralkyl group; R^{4VI} represents a C₈₋₁₈ alkyl group, a C₈₋₈ alkenyl group or a C₆₋₁₈ aralkyl group; and X^{VI-} represents an anion,

a compound of the formula (VII):

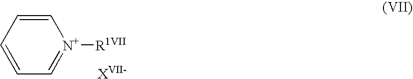

wherein R^{1VII} represents a C₈₋₁₈ alkyl group; and X^{VII-} represents an anion,

a compound of the formula (VIII):

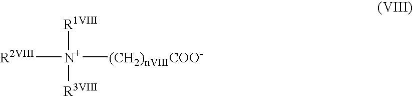

wherein R^{1VIII} and R^{2VIII} respectively represent a hydrogen atom, a C₁₋₈ alkyl group or a C₆₋₈ aralkyl group; R^{3VIII} represents a C₈₋₁₈ alkyl group, a C₈₋₁₈ alkenyl group or a C₆₋₁₈ aralkyl group; and nVIII represents 1 or 2, a compound of the formula (IX),

R^{1IX}—R^{2IX}—(CH₂CH₂O)_{nIX}—H (IX)

wherein R^{1IX} represents a C₉₋₂₅ alkyl group, a C₉₋₂₅ alkenyl group or a C₉₋₂₅ alkynyl group; R^{2IX} represents a group of formula

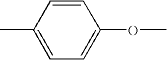

or —COO—; nIX represents from 10 to 40,

or a compound as listed below:

MEGA-8 of the Formula

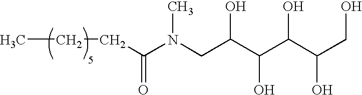

Sucrose Monocaprate:

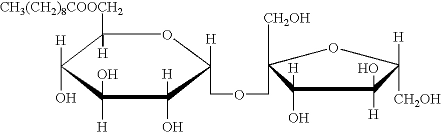

Deoxy-BIGCHAP:

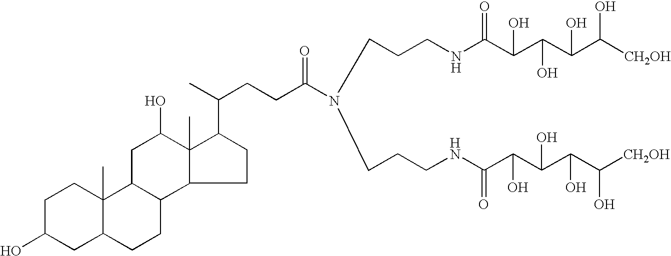

n-octyl-β-D-thioglucoside:

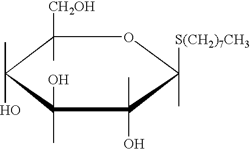

n-nonyl-β-D-thiomaltoside:

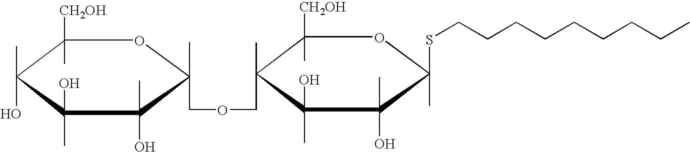

n-heptyl-β-D-thioglucoside:

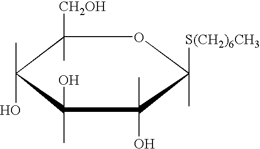

n-octyl-β-D-glucoside:

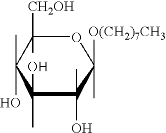

CHAPS:

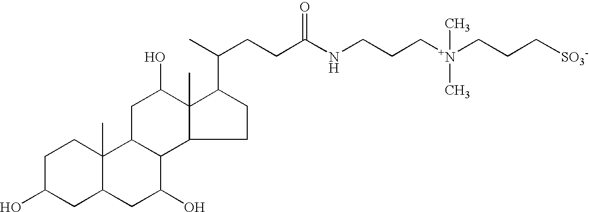

CHAPSO:

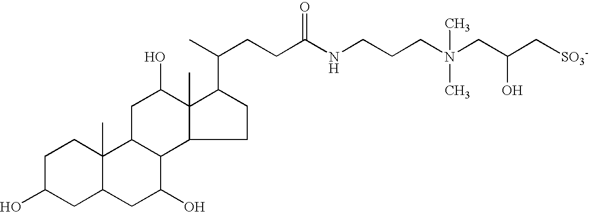

As the C₁₋₈ alkyl group, methyl, ethyl, propyl, t-butyl, n-butyl, isopentyl, neopentyl, t-pentyl, isohexyl, heptyl, octyl or the like is mentioned, among which C₁₋₃alkyl groups are preferred.

As the C₆₋₈ aralkyl group, benzyl, phenethyl or the like is mentioned.

As the C₈₋₁₈ alkyl group, octyl, decyl, dodecyl, tetradecyl, oleyl or the like is mentioned, among which C₁₀₋₁₈ straight-chain alkyl groups such as decyl, dodecyl, tetradecyl or the like are preferred.

As the C₆₋₁₈ alkenyl group, octenyl, decenyl, dodecenyl, tetradecenyl or the like is mentioned.

As the C₆₋₁₈ aralkyl group, phenylpropylene, phenylbutene, naphthylmethylene, naphthylethylene, naphthylpropylene, biphenylmethylene, biphenylethylene or the like is mentioned.

As the C₉₋₂₅ alkyl group, icosyl, henicosyl, tocosyl, tricosyl or the like in addition to the above-mentioned alkyl groups is mentioned.

As the C₉₋₂₅ alkenyl group, icosenyl, henicosenyl or the like in addition to the above-mentioned alkenyl groups is mentioned.

As the C₉₋₂₅ alkynyl group, icosynyl, henicosynyl or the like in addition to the above-mentioned alkenyl groups is mentioned.

The anion may be a halogen ion (fluorine ion, chlorine ion, bromine ion or iodine ion), halogenated boron ion (BF₄⁻, BCl₄⁻, BBr₄⁻, etc), phosphor compound ion, halogenated oxyacid ion, fluoro sulfuric ion, methylsulfuric ion, tetraphenyl boron compound ion having a halogen or halo alkyl substituent on the aromatic ring, among which bromine ion or BF₄⁻ is preferred.

Of the above-mentioned surfactants, those as listed from MEGA-8 to CHAPSO can be purchased from Kabushiki Kaisha Dojin Kagaku Kenkyusho, Japan.

The concentration of the surfactant used may be adjusted appropriately depending on the kind of the surfactant used and the components and concentrations of an erythrocyte lysing agent simultaneously used. Usually, if the concentration of the surfactant is too high, not only erythrocytes but also leukocytes and erythroid cells are excessively damaged. The shape of erythroid cells, in particular, is changed, which results in reduced differences in each intensity of fluorescence of erythroid cells, lipid particles and leukocytes as mentioned below. The concentration of the surfactant, therefore, is preferably about 10 to 10,000 mg/L, more preferably about 100 to 5,000 mg/L, furthermore preferably about 1000 to 3000 mg/L. It is noted that this concentration means the concentration of a surfactant in the first lysing agent. The sample of bone marrow fluid may be mixed with the first lysing agent suitably at 15 to 50° C., preferably at 20 to 40° C., suitably for 3 to 120 seconds, preferably for 5 to 40 seconds.

Subsequently, the sample of bone marrow fluid is stained with a fluorescent dye. It is necessary to use the first staining fluid containing at least one fluorescent dye which can produce a difference in each intensity of fluorescence at least of lipid particles, leukocytes and erythroid cells. The fluorescent dye may be selected from the following group: a compound of the formula (I):

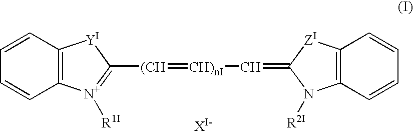

wherein R^{1I} and R^{2I} respectively represent a hydrogen atom, a lower alkyl group or lower alkenyl group optionally substituted by hydroxyl group; Y^{I} and Z^{I} respectively represent a sulfur atom, oxygen atom or nitrogen atom, or a carbon atom substituted with two lower alkyl groups; nI represents 0, 1 or 2; and X^{I-} represents an anion, a compound of the formula (II):

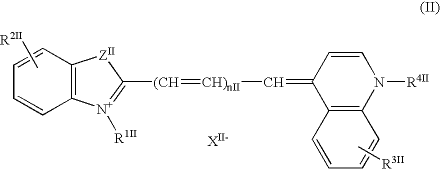

wherein R^{1II} represents a hydrogen atom or a lower alkyl group; R^{2II} and R^{3II} respectively represent a hydrogen atom, a lower alkyl group or a lower alkoxy group; R^{4II} represents a hydrogen atom, an acyl group or a lower alkyl group; Z^{II} represents a sulfur atom, oxygen atom or a carbon atom substituted with two lower alkyl groups; nII represents 0, 1 or 2; and X^{II-} represents an anion,

a compound of the formula (III):

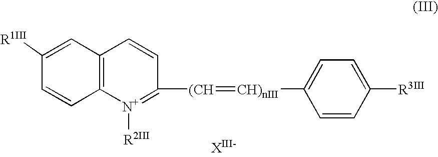

wherein R^{1III} represents a hydrogen atom or a dimethylamino group; R^{2III} represents a lower alkyl group; R^{3III} represents a hydrogen group or a dimethylamino group; nIII represents 1 or 2; and X^{III-} represents an anion,

a compound of the formula (IV)

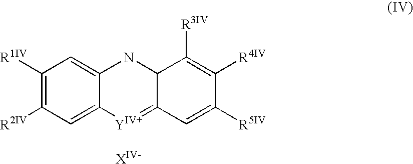

wherein R^{1IV} represents a hydrogen atom or a lower alkyl group; R^{2IV} represents a dimethylamino group; R^{3IV} represents a hydrogen atom or an amino group; R^{4IV} represents a hydrogen atom, a lower alkyl group or an amino group; R^{5IV} represents a hydrogen atom or a dimethylamino group; X^{IV-} represents an anion; and Y^{IV} represents a sulfur atom or an oxgen atom,

a compound of the formula (V):

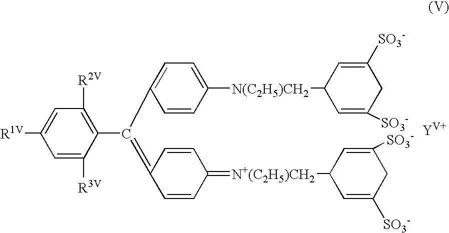

wherein R^{1V} represents a hydrogen atom or a hydroxyl group; R^{2V} represents a hydrogen atom or a sulfonic group; R^{3V} represents a hydrogen atom or a sulfonic group; and Y^{V+} represents an alkali metal cation,

and the compounds of the following formula:

NK-2825:

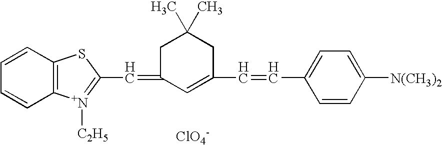

NK-1836:

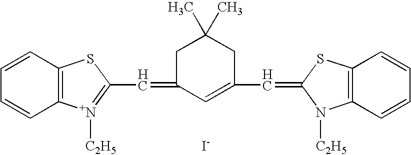

NK-1954:

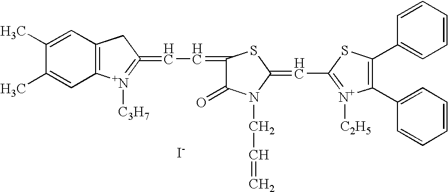

Oxazine750:

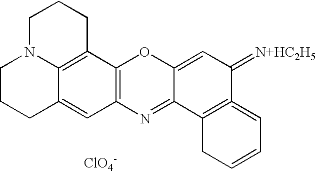

Cryptocyanine:

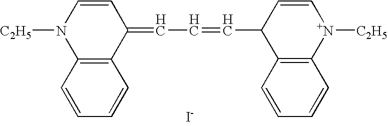

NK-376:

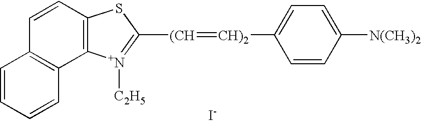

NK-382:

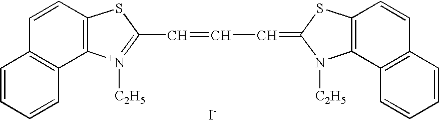

NK-2711:

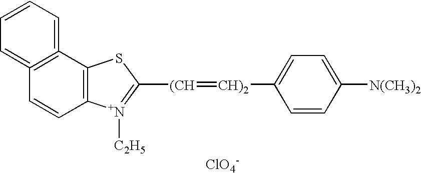

NK-138:

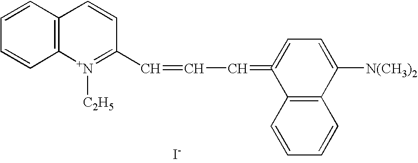

Oxazine720:

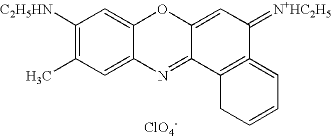

LDS730:

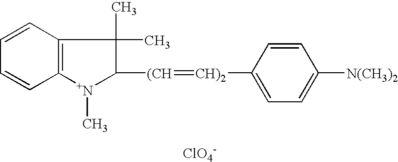

LD700:

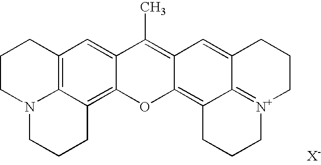

Nile Blue A:

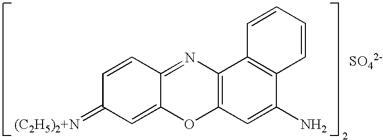

Brilliant Green:

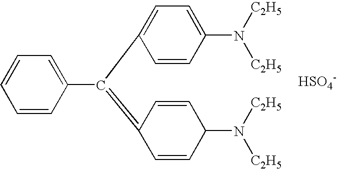

Iodide Green:

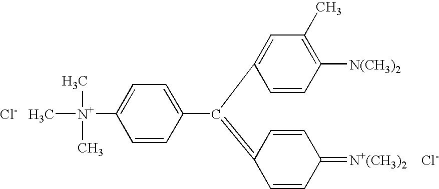

and

Malachite Green:

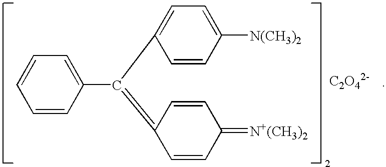

In the above formulae, alkyl groups bound to nitrogen atoms or carbon atoms of heterocycles are straight-chain or branched alkyl groups having 1 to 20, preferably 1 to 10, more preferably 1 to 6 carbon atoms, including methyl, ethyl, propyl, t-butyl, n-butyl, n-pentyl, n-hexyl and the like, for example.

The lower alkyl group means a straight-chain or branched alkyl group having 1 to 8 carbon atoms, and examples thereof include methyl, ethyl and the like.

The lower alkoxy group means a straight-chain or branched alkoxy group having 1 to 8 carbon atoms, and examples thereof include methoxy, ethoxy and the like.

The lower alkynyl group means an alkynyl group having 2 to 5 carbon atoms, and examples thereof include ethynyl, propynyl, butynyl, pentynyl and the like.

Acyl groups may be mentioned those having 1 to 3 carbon atoms, including formyl, acetyl, propionyl and the like.

Anions may be mentioned halogen ions such as F⁻, Cl⁻, Br and I⁻, CF₃SO₃⁻, BF₄⁻, ClO₄⁻ and the like.

Alkali metal cations may be mentioned Li⁺ Na⁺, K⁺, and the like.

Of the above-mentioned dyes, the NK series can be purchased from Nippon Kanko Shikiso Kenkyusho Kabushiki Kaisha, Japan, LDS730 and LD700 can be purchased from Exciton Company, and the others are commercially available.

After preceding the step for the erythrocyte dissolution or the like with the first lysing agent, the fluorescent dye is used by dissolving in a proper solvent (e.g. water, lower alcohol, ethylene glycol, DMSO, etc.). Alternatively, it may be dissolved in the first lysing agent and allowed to act on (mixed with) the sample of bone marrow fluid simultaneously together with the first lysing agent.

In the present invention, as a preferable embodiment in step where the sample of bone marrow fluid is mixed and stained with the first lysing agent together with the first staining solution, an organic acid such as salicylic acid, dyes and surfactants are dissolved with purified water and the solution obtained is adjusted at appropriate pH with use of NaOH and HCl and the like. The resulting solution is mixed with the sample of bone marrow fluid and reacted suitably at 15 to 50° C., preferably at 20 to 40° C., suitably for 3 to 120 seconds, preferably for 5 to 40 seconds.

The concentration of the dye used may vary depending on the kind of the dye used, but may be generally 0.01 to 100 mg/L, preferably 0.1 to 10 mg/L, more preferably 0.3 to 3.0 mg/L. This concentration of the dye means a concentration in a mixture of the sample of bone marrow fluid, the first lysing agent and the fluorescent dye.

By this staining, leukocytes are strongly stained and emit fluorescence with strong intensity. Erythroid cells are weakly stained and emit fluorescence with weak intensity. Lipid particles, if contained, are also weakly stained and emit fluorescence with weak intensity. The mechanism of producing a difference in each intensity of fluorescence of leukocytes and of erythoid cells is not clearly known. However, probably since the nuclei (DNAs) of erythroid cells shrink, a dye is hindered from being taken into the nuclei of the cells, but the mechanism is not clearly elucidated.

Apart from the step (2)(i), in the step (2)(ii), the second lysing agent is mixed with the sample of bone marrow fluid. Thereby, leukocytes except immature myeloid cells contained in the sample of bone marrow fluid are damaged. Normal leukocytes possess the substance-eliminating function eliminating unnecessary substance (e.g. dyes). The damage of leukocytes usually means a mechanism of which a dye, not passing through the cell membranes of living leukocytes is permeable into the cell membrane. To put it concretely, when a lysing agent of particular components acts on the cells, pores are made in the cell membranes which particular substances can be only passed through the cell membrane by extracting (pulling out) a part of lipid components of particular cell (e.g. normal leukocytes). The mechanism is not clearly elucidated. Dye molecules possibly pass through cell membrane via the pores, and as the result, can permeate into the particular cells and stain them. While, immature myeloid cells are not stained with a dye because pores enough to pass through dye are made.

In the present invention, leukocytes are classified into lymphoid cells, mature myeloid cells and immature myeloid cells. Among them, lymphoid cells mean mature lymphocytes, immature lymphocytes and a typical lymphocytes. Mature myeloid cells mean mature monocytes and granulocytes. Immature myeloid cells mean immature myeloid leukocytes which are usually present in the bone marrow and do not present in the peripheral blood; for example, myeloblasts, promyelocytes, myelocytes, metamyelocytes. Myeloid blasts mean myeloblast and monoblast. Promyelocytes, myelocytes and metamyelocytes in combination may be also defined as immature granulocytes. While, myeloid blast and immature granulocytes in combination may be called as immature myeloid cells. Furthermore, hematopoietic precursor cells such as myeloid stem cell (CFU-GEMN), granulocytes, macrophage-colony forming unit (CFU-GM), eosinocyte-colony forming unit (CFU-EOS) and others in the differentiation state of preblasts also are involved in immature myeloid cells. Mature myeloid cells and mature lymphocytes in combination are defined as mature leukocytes.

The second lysing agent used in the present invention preferably comprises a surfactant, a solbilizer, an amino acid and a buffer.

The surfactant may be used various kinds of surfactants, such as may be a compound of the following formula (XI):

R^{1XI}—R^{2XI}—(CH₂CH₂O)_{nXI}—H (XI)

wherein R^{1XI} represents a C₁₀₋₂₅ alkyl group, a C₁₀₋₂₅ alkenyl group or a C₁₀₋₂₅ alkynyl group; R^{2XI} represents the following formula

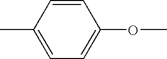

, or

—COO—; n_{XI} represents from 10 to 40

The C₁₀₋₂₅ alkyl groups may be mentioned decyl, undecyl, dodecyl, tridecyl, tetradecyl and the like.

The C₁₀₋₂₅ alkenyl group may be mentioned dodecenyl, tetradecenyl and the like.

The C₁₀₋₂₅ alkynyl group may be mentioned dodecynyl, undecynyl, dodecynyl and the like.

Preferred examples thereof include polyoxyethylene (20) laurylether, polyoxyethylene (15) oleylether, and polyoxyethylene (16) oleylether.

The concentration of the surfactant used depends on the kind of the surfactant used, but as polyoxyethylene nonion surfactant, if carbon number of hydrophobic group is the same, the more smaller number of n^{XI} make the more stronger power of damaging for the cells, the larger number of n^{XI} make the less strong power of damaging the cells. When the number of n^{XI} is the same, carbon less number of hydrophobic groups make the more strong power of damaging for a cell. Take account of this point, the required concentration of surfactant can be simply calculated by examination.

Examples thereof may be used the range from 0.1 to 2.0 g/L (preferably from 0.5 to 1.5 g/L) for the said polyoxyethylene (20) laurylether, from 1 to 9 g/L (preferably 3 to 7 g/L) for polyoxyethylene (15) oleylether, from 5 to 50 g/L (preferably from 15 to 35 g/L) for polyoxiethylene (16) oleylether.

The second lysing agent may contain as the solubilizing agent at least one or more and is selected from the following compounds:

a sarcosine derivative or salt thereof of the formula (XII):

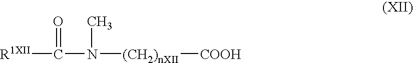

wherein R^{1XII} represents a C₁₀₋₂₂ alkyl group; n_{XII} represents from 1 to 5,

a cholic acid derivative of the formula (XIII):

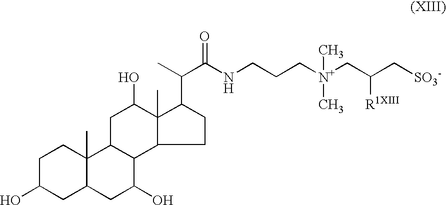

wherein R^{1XIII} represents a hydrogen atom or hydroxyl atom, and

a methylglucan amide of the formula (XIV)

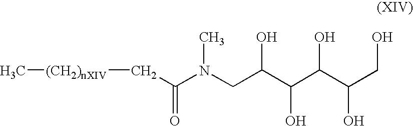

wherein n_{XIV} represents from 5 to 7.

The C₁₀₋₂₂ alkyl groups may be decyl, dodecyl, tetradecyl, oleyl and the like.

Specifically, the solubilizing agent suitably used are sodium N-lauroyl sarcosinate sodium lauroyl methyl β-alanine, lauroyl sarcosine, CHAPS (3-[(3-cholic amide propyl)dimethyl ammonio]-1-propane sulfonate), CHAPSO ([(3-cholic amide propyl)dimethyl ammonio]-2-hydroxy-1-propane sulfonate), MEGA8 (octanoyl-N-methylglucan amide), MEGA9 (nonanoyl-N-methyl glucamide), MEGA1 (decanoyl-N-methyl glucamide), and the like.

The concentration of the solubilizing agent is preferably 0.2 to 2.0 g/L for sarcosine derivative or salt thereof, 0.1 to 0.5 g/L for cholic acid derivatives, 1.0 to 8.0 g/L for methylglucan amide.

Other solubilizing agents may be n-octyl β-gulcoside, sucrose monocaprate, N-formylmethylleucinealanine, and the like, preferably for the concentration thereof from 0.01 to 50.0 g/L.

The amino acid in the second lysing agent may be amino acids constituting proteins, preferably glutamic acid, valine and sulfur containing amino acid such as methionine, cystine and cysteine, more preferably methionine. The amount used ranges from 1 g/L to 50 g/L; suitably 8 g/L to 12 g/L for glutamic acid and suitably 16 g/L to 24 g/L for methionine.

The buffer in the second lysing agent may be Good buffer solution like HEPES and phosphoric buffer, where a pH adjusting agent such as sodium hydroxide and, if necessary, an osmotic pressure adjusting agent such as sodium chloride are added to make a pH and an osmotic pressure of 5.0 to 9.0, and 150 to 600 m Osm/kg.

A preferable one as the second lysing agent is the agent as described in U.S. Pat. No. 5,413,938 which contains (1) nonionic surfactant of polyoxyethylenes for solidifying cytoplasm and cell membranes of the immature myeloid cells; (2) solubilizing agent for damaging the cell membranes of other hematocytes to shrink them; (3) amino acid for solidifying the cytoplasm and cell membranes of immature myeloid cells; (4) buffer which renders the solution adjusted to pH 5.0 to 9.0 and osmotic pressure to 150 to 600 m Osm/kg

To produce a difference in intensity of fluorescence between the first group containing the mature myeloid cells and lymphocytes and the second groups containing immature myeloid cells, the fluorescent dye in the second staining solution of the present invention may be sufficient in practical use if either the damaged cells or immature myeloid cells can be stained. The fluorescent dye for staining the damaged cells may be any dye which is specific to the cell nuclei especially to DNA or RNA. Some cationic dyes are suitable for use.

In general, cationic dyes pass through the cell membranes of living cells and stain the components in the cells. However, it is well known that specific cationic dyes (e.g. ethidium bromide, propidium iodide, et.al) do not pass through the living cells and stain only the damaged cells.

Specifically, the fluorescence dye in the second staining solution may be said ethidium bromide and propidium iodide and ethidium-acridine heterodimer which can be purchased from molecular probe, ethidium azide, ethidium homodimer-1, ethidium homodimer-2, ethidium monoazide, TOTO-1, TO-PRO-1, TOTO-3, TO-PRO-3, and the like

Furthermore, when He—Ne, red semiconductor laser as a source light is used, a suitable dye is the following formula (X):

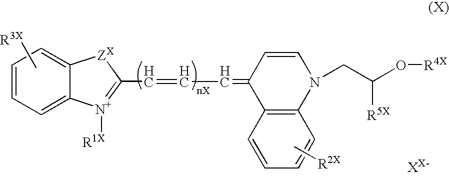

wherein R^{1X} represents a hydrogen atom or a lower alkyl group; R^{2X} and R^{3X} respectively represent a hydrogen atom, a lower alkyl group or a lower alkoxyl group; R^{4X} represents a hydrogen atom, an acyl group or a lower alkyl group; R^{5X} represents a hydrogen atom or a lower alkyl group optionally substituted; Z^{X} represents a sulfur atom, an oxygen atom or a carbon atom substituted with two lower alkyl groups; n_{X} represents 1 or 2; X^{X-} represents an anion.

The lower alkyl group in R^{1X}, R^{2X}, R^{3X}, R^{4X}, R^{5X}, and Z^{X} of said formula means a straight-chain or branched C₁₋₆ alkyl group, such as methyl, ethyl, propyl, butyl, isobutyl, sec-butyl, ter-butyl, pentyl, hexyl group, among which methyl and ethyl group are preferred.

The lower alkoxyl group in R^{2X} and R^{3X} means C₁₋₆ alkoxyl group, and may be mentioned such as methoxy, ethoxy, propoxy, and like, among which methoxy group and ethoxy group are preferable.

The lower acyl group in R^{4X} is preferably an acyl group, derived from aliphatic carboxylic acid, such as acetyl, propionyl and the like, preferably acetyl. The lower alkyl group is the same as mentioned above.

The lower alkyl group optionally substituted in R^{5X} means a lower alkyl group optionally substituted by 1 to 3 of hydroxyl, halogen (fluorine, chlorine, bromine or iodine) and the like, among which methyl or ethyl substituted by one hydroxyl is preferable.

Z^{X} is preferably a sulfur atom.

The anion in X^{X-} may be a halogen ion (fluorine, chlorine, bromine or iodine ion), halogenated boron ion (BF₄⁻, BCl₄⁻, BBr₄⁻, etc), phosphorous compound ion, halogenated oxyacid ion, fluoro sulfuric ion, methyl sulfuric ion, tetraphenyl boron ion having a halogen or a halogenated alkyl group as substituent in the aromatic ring, among which bromine ion or BF₄⁻ is preferable.

Specific example of the dyes is the following dyes, without limiting thereto.

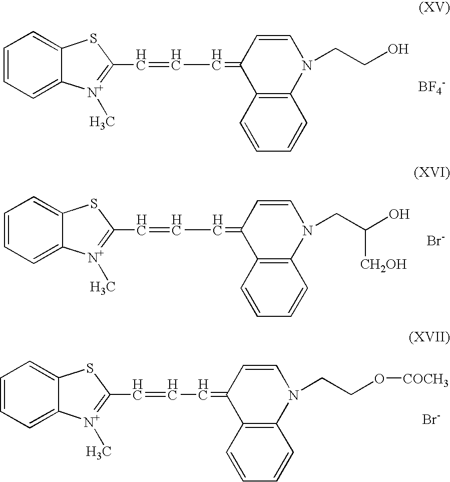

As a preferable embodiment for step (2) where the sample of bone marrow fluid is mixed stained with the second lysing agent the second staining solution, the sample is mixed with the second lysing agent in which a dye to be contained in the second staining solution is added. That is, the second lysing agent is prepared by dissolving the dye therein or by mixing a solution of the dye in a water soluble organic solvent as ethylene glycol when used. This mixing when used is preferable because of providing increased stability of the dye. The concentration of the dye is appropriately determined depending on the dye used. For example, it is 0.01 to 100 mg/L, preferably 0.1 to 30 mg/L for ethidium bromide.

The mixing of the sample of bone marrow fluid and the second lysing agent containing dye can be suitably conducted at the ratio of the sample to the second lysing agent containing the dye being 1:10 to 1:100, at 20 to 40° C., and for 5 second to 5 minutes. It is preferable that when the reaction temperature is high, the reaction time be made short.

In step (3), each sample prepared in step (2)(i) or (ii) is introduced to a flow cytometer to measure at least one kind of scattered light and at least one kind of fluorescence. Any flow cytometer which is commercially available can be used.

In the present invention, the scattered light means scattered light that can-be measured by a commercially available flow cytometer, and includes forward low-angle scattered light (0° to less than 5° as an example of incident light angle), forward high-angle scattered light (about 5° to 20° as an example of incident light angle), and side scattered light (about 90° as an example of incident light angle). Preferably, the side scattered light is selected, which reflects intercellular informations such as the nuclear form of cells.

Fluorescence is emitted from the said dye having stained the cell components. A suitable received light wavelength is selected according to the dye used. Fluorescent signals reflect cytochemical properties.

The light source of flow cytometer is not particularly limited, but one having a wavelength suitable for exciting the dye is selected. For example, an argon ion laser, a He—Ne laser, a red semiconductor laser and others may be used. The semiconductor laser, especially, is far less expensive than gas lasers and can contribute to a great reduction in the cost of flow cytometer.

In step (4), cells are classified and counted using a difference in each intensity of the measured scattered light and fluorescence of the sample stained by step (2)(i) or (ii).

For example, in step (4)(i), lipid particles, erythroid cells and leukocytes are classified and counted using a difference in each intensity of the scattered light and fluorescence of the sample stained in step (2)(i).

For “classifying and counting the lipid particles, erythroid cells and leukocytes using a difference in each intensity of the scattered light and fluorescence”, for example, at first, a scattergram is depicted with each intensity of the fluorescence in X axis and that of the side scattered light in Y axis respectively.

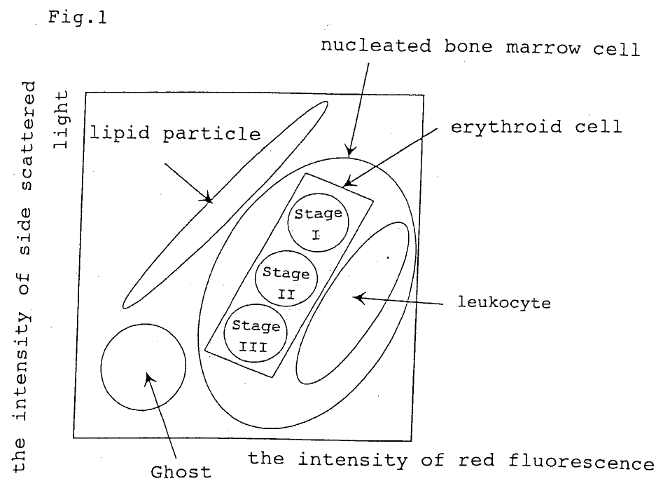

In this case, as shown in FIG. 1, each of erythroid cells, leukocytes, lipid particles and ghost are distributed to form their respective clusters. Next, using an appropriate analysis software, a region is set for each cluster and cell count in the cluster is calculated. Thereby, it is possible to classify and count lipid particles, erythroid cells and leukocytes.

From the obtained erythroid cell count and leukocyte count, it is possible to calculate the ratio of leukocytes to erythroid cells. Then, nucleated cell count of bone marrow can be calculated from putting together leukocyte count and erythroid cell count.

The erythroid cells may be classified and counted into at least two groups according to their degree of maturity using a difference in each intensity of the scattered light and fluorescence of the sample stained in step (2)(i). For analyzing erythroid cells according to their degree of maturity, a scattergram is produced substantially as described above, a region is set for each group according to the degree of maturity, and the cells in the region are counted. Thereby, it is possible to calculate the ratio of erythroblasts at each degree of maturity to all the erythroid cells from the erythroid cell count and the erythroid cell count at each degree of maturity.

The ratio of myeloid cells to erythroid cells is usually 1.5:1 to 3.3:1. Diseases such as leukemia change these ratios. These ratios then are useful for diagnosing acute myelocytic leukemia (AML) and myelodysplastic syndrome (MDS). Therefore, by measuring these ratios with times, it is possible to grasp the pathology of various kinds of leukemia, to monitor the treatment and others.

Furthermore, it is possible to grasp the state of erythropoiesis in the bone marrow from the erythroid cell count in nucleated cells of bone marrow.

In step (4)(ii), mature myeloid cells, lymphoid cells and immature myeloid cells are classified and counted using the measured scattered light and fluorescence of the sample stained in step (2)(ii).

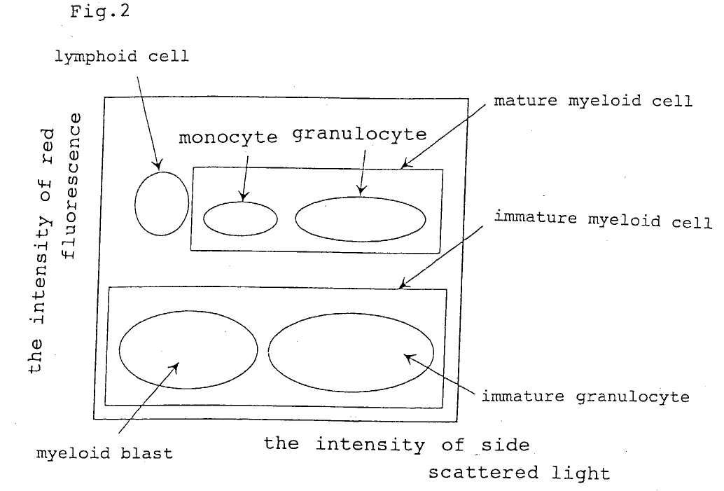

For “to classify and count mature myeloid cells, lymphoid cells and immature myeloid cells with each intensity of scattered light and fluorescence measured”, for example, a scattergram is depicted using fluorescence in X axis and side sscattered light in Y axis. In this case, as shown in FIG. 2, mature myeloid cells, lymphoid cells and immature myeloid cells are distributed to form their respective clusters. Next, using an appropriate analysis software, a region is set for the region of each cluster, and cell count in the region is calculated. Thereby, it is possible to classify and count the mature myeloid cells, lymphoid cells and immature myeloid cells.

Furthermore, in step (4)(iii), myeloid cell count can be calculated by putting together the above-mentioned mature myeloid cell count and immature myeloid cell count. The ratio of myeloid cells to erythroid cells (M/E ratio) is thus also calculated from the erythroid cell count and myeloid cell count obtained.

Mature myeloid cells are classified and counted into at least two groups using a difference in each intensity of scattered light and fluorescence of the sample stained in step (2)(ii). For classifying and counting mature myeloid cells, a scattergram is practically illustrated as mentioned above, and as shown in FIG. 2, a region is set for each cluster of monocytes and granulocytes, and cells in a region are counted.

In addition, immature myeloid cells are classified and counted into at least 2 groups using a difference in each intensity of scattered light and fluorescence of the sample stained in step (2)(ii). For immature myeloid cells, a scattergram is depicted as mentioned above, and a region is set for each cluster of myeloid cells and immature granulocytes, as shown in FIG. 2, and cells in the cluster are counted.

In the method of the present invention, steps (2)(i) and (2)(ii) can be conducted in any order or simultaneously. Futhermore, the same is said for steps (4)(i) and (4)(ii).

The present invention provides an automatic analyzer for analyzing nucleated cells of bone marrow fluid comprising:

a dispenser for preparing a first and second samples from a sample of bone marrow fluid;

a first reaction chamber for preparing a first measuring sample, the first measuring sample being obtained by mixing the first sample with a first lysing agent to lyse erythrocytes in the first sample, and mixing the mixture with a first staining solution containing at least a first fluorescent dye for producing a different in each intensity of fluorescence among at least leukocytes, erythroid cells and lipid particles;

a second reaction chamber for preparing a second measuring sample, the second measuring sample being obtained by mixing the second sample with a second lysing agent to damage all the cells but immature myeloid cells, and mixing the mixture with a second staining solution containing at least a second of fluorescent dye for producing a different in each intensity of fluorescence between a group of mature myeloid cells and lymphoid cells and a group of immature myeloid cells;

a flow cytometer for measuring a scattered light and a fluorescence of the first measuring sample and the second measuring sample; and

a data processor for obtaining a ratio of myeloid cells and erythroid cells (M/E ratio) by classifying and counting leukocytes, erythroid cells and lipid particles with use of a difference in each intensity of the scattered light and the fluorescence of the first measuring sample, classifying and counting mature myeloid cells, lymphoid cells and immature myeloid cells with use of difference in each intensity of the scattered light and the fluorescence of the second measuring sample, calculating the number of myeloid cells from the counted mature myeloid cells and immature myeloid cells, and calculating the counted erythroid cells and myeloid cells.

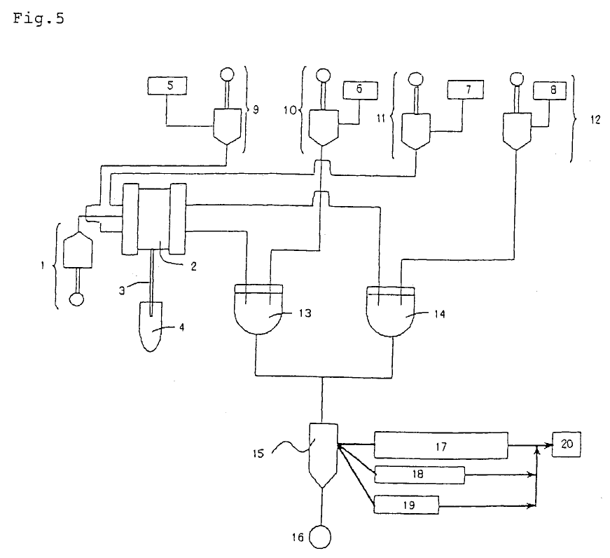

FIG. 5 schematically shows the automatic analyzer for analyzing nucleated cells of bone marrow fluid. The dispenser is composed of an aspiration pump (1), a sampling valve (2), and an aspiration pipette for a sample (3). The reaction chamber is composed of a first chamber (13) and a second one (14). The flow cytometer consists of a flow cell (15), a detector for forward (low-angle or high-angle) scattered light (17), a detector for side scattered light (18), a detector for fluorescence (19) and a data processor (20). The supplier is composed of a first lysing agent container (5), a first staining solution container (6), a second lysing agent container (7) a second staining solution container (8) and aspiration-emission pumps (9)˜(12) connected with each container via tubes. A negative pressure pump (16) can be employed as the introduction means.

In practical, the sample of bone marrow fluid in test tube (4) is introduced to the sampling valve (2) through the tube using aspiration pump from the aspiration pipette (3) aspirating the sample. The appropriate amount is quantitatively collected. The quantitatified sample of bone marrow fluid is introduced into the first reaction chamber (13) via the sampling valve (2) or into the second chamber (14) via the aspiration-emission valve (9) or (11) via tube.

The sampling valve stated in the present invention is defined to overlap three of round-form discs such as made of ceramic. The sampling valve consists of a mobile element sandwiched between two fixed elements, in which a flown-in route and/or a flow-out route are set for the fixed elements, and a quantitative analysis route is set for the mobile element. The sample is passed through the flow-in route, quantification route and flow-out route in order. For the fixed elements, the reagent flow-in route and/or the flow-out route are set, and the mobile element is rotated to pass the reagent into the flow-in route, quantification route and flow-out route in order. Next, the quantified sample is flowed out into the reaction chamber with the reagent. Such a sampling valve is used in the analytical apparatus for performing blood assay.

To the first reaction chamber (13), the first lysing agent collected from the first lysing agent container (5) with the aspiration-emission pump (9) is, via the tube, introduced through the sampling valve (2) with the sample of bone marrow fluid, and is mixed with the sample of bone marrow fluid in the chamber (13). Thereafter, the first staining solution collected in appropriate amount from the first staining solution container (6) with the aspiration-emission pump (10) is introduced to the said chamber (13) to stain leukocytes and others.

The sample obtained in such steps is introduced from the chamber (13) to the flow cell (15) via the tube with use of the negative pressure pump (16), and scattered light and fluorescence were measured. After measurement, the data for scattered light and fluorescence which are detected in the detector for forward (low-angle or high-angle) scattered light (17), the detector for side scattered light (18) and the detector for fluorescence (19) were assayed in the detector. Then, nucleated cells of bone marrow fluid are classified and counted.

In the second reaction chamber (14), the sample is treated with the second lysing agent and the second staining solution which were introduced from the container of the second lysing agent (7), and the container of the second staining agent, and measured in the flow cell (15). The obtained data is analyzed in the data processor (20), and nucleated cells of bone marrow fluid are classified and counted to calculate the M/E ratio.

In the analyzer of the present invention, the data processor (20) comprises a computer made up of a CPU, ROM, RAM and I/O port, or a personal computer.

The present invention is now described in detail by way of the following example. It should be constructed that various changes and modifications may be made to the present invention and that the scope of the present invention is not limited to the example.

Embodiment

Reagents having the following compositions were prepared.

| | |
|---|---|
| First lysing agent: | |
| Salicylic acid (commercially available) | 10 mM |
| LTAC (dodecyltrimethylammonium chloride) | 0.3 g/L |
| (commercially available) | |
| Purified water | 1 L |
| Adjusted to pH 3.0 with NaOH (an osmotic | |
| pressure of 40 mOsm/Kg). | |
| First staining solution: | |
| NK-2825 (Nippon Kanko Shikiso Kenkyusho) | 0.3 mg/L |
| in ethylene glycol | |
| Second lysing agent: | |
| Polyoxyethylene (16) oleylether | 24.0 g/L |
| N-sodium lauroyl sarcosinate | 1.5 g/L |
| DL-methionine | 20.0 g/L |
| HEPES | 12.0 g/L |
| 1 N-NaCl | 0.3 g/L |
| Second staining solution: | |
| Dye of formula (XV) | 3.0 mg/L |
| in ethylene-glycol | |

Bone marrow fluid of patients with acute myelocytic leukemia (AML) in the test tube (4) was introduced to the sampling valve (2), through the aspiration pipette aspirating by the aspiration pump (1) via the tube, and 30 μl (sample No. 1) and 33 μl (sample No. 2) of the bone marrow fluid were assayed quantitatively. Sample No. 1 was introduced with 0.98 ml of the first lysing agent to the first reaction chamber (13) with use of the aspiration-emission pump (5). While, sample No. 2 was introduced with 0.95 ml of the second lysing agent to the second reaction chamber (14) with use of the aspiration-emission pump (11).

Next, 0.02 ml of the first staining solution was added to the sample adjusted in the first reaction chamber (13) by using the aspiration-emission pump (10), and allowed to react at 40° C. for 5 seconds. Thereafter, side scattered light and fluorescence were measured in the flow cell. While, 0.05 ml of the second staining fluid was added to the sample prepared in the second reaction chamber (14) with use of the aspiration-emission pump (12), and allowed to react at 33° C. for 10 seconds. Then, side scattered light and (red) fluorescence were measured in the flow cell. As a light source, a red semiconductor laser of 633 nm was used, and fluorescence having a wavelength of 660 nm or more was measured. After the measurement, the data for scattered light and fluorescence which were detected in the detector for forward (low-angle or high-angle) scattered light (17),the detector for side scattered light (18) and the detector for fluorescence were analyzed in the data processor (20).

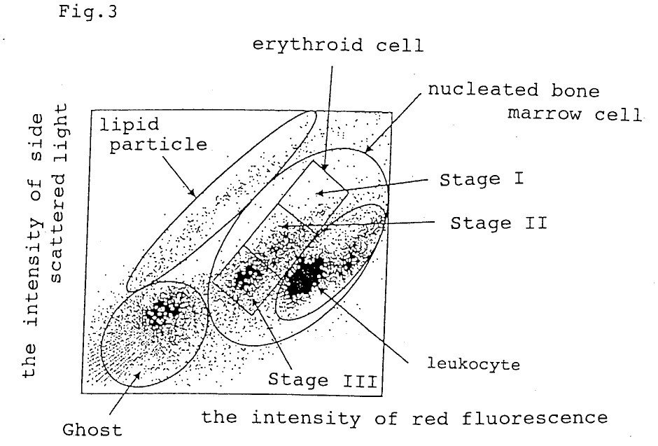

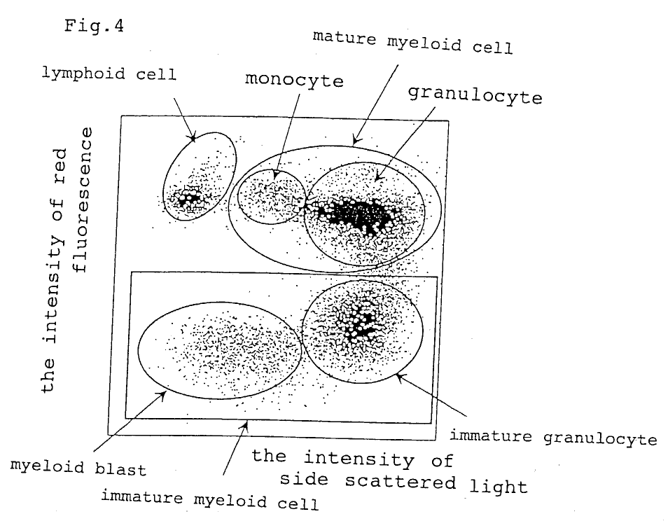

FIG. 3 is a scattergram with each intensity of red fluourescence and each intensity of side scattered light plotted in the abscissa and in the ordinate, respectively. FIG. 4 shows a scattergram with each intensity of side scattered light and each intensity of red fluorescence allotted in the abscissa and in the ordinate, respectively. FIG. 3 indicates the five clusters, i.e., of leukocytes, of erythroid cells in stage I, of erythroid cells in stage II, of erythroid cells in stage III and lipid particles. FIG. 4 shows the five clusters, i.e., of lymphoid cells, of monocytes, of granulocytes, of immature granulocytes and of myeloblasts.

The above bone marrow fluid was subjected to May-Grünwald's stain and was then microscopically observed. Leukocytes and erythroid cells were classified, and erythroid cells were further differenticated into proerythroblasts, basophilic erythroblasts, polychromatic erythroblasts, orthochromatic erythroblasts. Furthermore, lymphoid cells, monocytes, immature granulocytes and myeloblasts were classified. The ratio of erythroid cells to myeloid cells was calculated and compared with the said results obtained by the flow cytometer.

Table 1 shows the flow-cytometrically obtained results and the microscopically obtained results.

TABLE 1

| | claimed | microscopical |
|---|---|---|
| | method | method |
| nucleated bone marrow cell | 1149 | 1075 |
| count(×10³/μl) | | |
| leukocyte count(×10³/μl) | 1035 | 970 |
| erythroid cell count(×10/μl) | 114 | 105 |
| erythroid cells in stage I | 0.5% | — |
| erythroid cells in stage II | 17.8% | — |
| erythroid cells in stage III | 81.7% | — |
| leukocyte/erythroid cell ratio | 9.1:1 | 9.2:1 |
| lymphoid cell count | 155 | 126 |
| monocyte count(×10³/μl) | 52 | 68 |
| granulocyte count(×10³/μl) | 466 | 388 |
| granular leukocyte count(×10³/μl) | 310 | 340 |
| myeloid blast count(×10³/μl) | 52 | 49 |
| myeloid cell count(×10³/μl) | 880 | 845 |
| M/E ratio | 7.7:1 | 8.0:1 |

As the results obtained microscopically, basophilic erythrocytes and proerythroblasts are 0%, polychromatic erythroblasts are 18%, and orthochromatic erythroblasts are 82%. This finding coincides well with the classified data in erythroblasts in stage I, erythroblasts in stage II and erythroblasts in stage III. Table 1 shows that both the results agree considerably well with each other.

As mentioned above, the technique in the present invention provides a method for the calculation of M/E ratio in a simple procedure and high precision.

1. A method for automatically analyzing nucleated bone marrow cells comprising the steps:
(1) preparing a first and second samples from a sample of bone marrow fluid;
(2) (i) preparing a first measuring sample obtained by mixing the first sample with a first lysing agent to lyse erythrocytes in the first sample, and mixing the mixture with a first staining solution containing at least a first fluorescent dye for producing a difference in each intensity of fluorescence among at least leukocytes, erythroid cells and lipid particles;
(ii) preparing a second measuring sample obtained by mixing the second sample with a second lysing agent to damage all the cells but immature myeloid cells, and mixing the mixture with a second staining solution containing at least a second fluorescent dye for producing a difference in each intensity of fluorescence between a group of mature myeloid cells and lymphoid cells and a group of immature myeloid cells;
(3) introducing each of the first and second measuring samples to a flow cytometer to measure at least one kind of scattered light and at least one kind of fluorescence;
(4) (i) classifying and counting leukocytes, erythroid cells and lipid particles with use of a difference in each intensity of the scattered light and the fluorescence of the first measuring sample;
(ii) classifying and counting mature myeloid cells, lymphoid cells and immature myeloid cells with use of a difference in each intensity of the scattered light and fluorescence of the second measuring sample;
(iii) calculating a number of myeloid cells from the counted mature myeloid cells and immature myeloid cells;
(5) calculating a ratio of myeloid cells and erythroid cells (M/E ratio) from the counted erythroid cells and myeloid cells.

2. A method as claimed in claim 1, which further comprises classifying and counting erythroid cells at each degree of maturity into at least two groups using a difference in each intensity of the scattered light and the fluorescence of the first measuring sample, and calculating a ratio of erythroblasts at each degree of maturity to all the erythroid cells from the erythroid cell count and the erythroid cell count at each degree of maturity.

3. A method as claimed in claim 1, which further comprises calculating nucleated bone marrow cell count from the obtained leukocyte count and the erythroid cell count.

4. A method as claimed in claim 1, which further comprises calculating a ratio of leukocytes to erythroid cells from the obtained erythroid cell count and leukocyte count.

5. A method as claimed in claim 1, which further comprises classifying and counting mature myeloid cells into at least two groups using each intensity of the scattered light and the fluorescence of the second measuring sample.

6. A method as claimed in claim 1, which further comprises classifying and counting immature myeloid cells into at least two groups using each intensity of the scattered light and fluorescence of the second measuring sample.

7. A method as claimed in claim 1, wherein the first fluorescent dye is one or more dyes selected from the following group:
a compound of the formula (1): 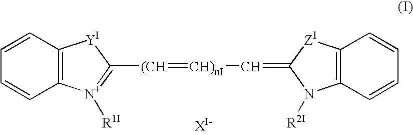
wherein R^{1I} and R^{2I} respectively represent a hydrogen atom, a lower alkyl group or lower alkenyl group optionally substituted by hydroxyl group; Y^{I} and Z^{I} respectively represent a sulfur atom, oxygen atom, or nitrogen atom, or a carbon atom substituted with two lower alkyl groups; nI represents 0, 1 or 2; and X^{I-} represents an anion,
a compound of the formula (II): 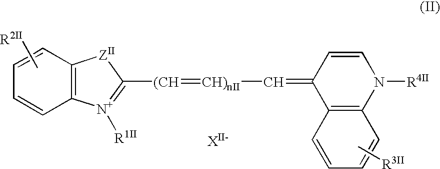
wherein R^{1II} represents a hydrogen atom or a lower alkyl group; R^{2II} and R^{3II} respectively represent a hydrogen atom, a lower alkyl group or a lower alkoxy group; R^{4II} represents a hydrogen atom, an acyl group or a lower alkyl group; Z^{II} represents a sulfur atom, oxygen atom or a carbon atom substituted with two lower alkyl group; nII represents 0, 1 or 2; and X^{II-} represents an anion,
a compound of the formula (III): 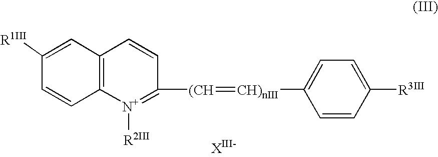
wherein R^{1III} represents a hydrogen atom or a dimethylamino group; R^{2III} represents a lower alkyl group; R^{3III} represents a hydrogen group or a dimethylamino group; nIII represents 1 or 2; and X^{III-} represents an anion,
a compound of the formula (IV) 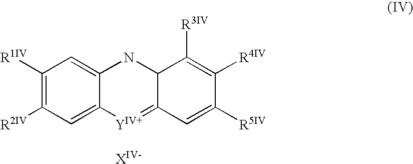
wherein R^{1IV} represents a hydrogen atom or a lower alkyl group; R^{2IV} represents a dimethylamino group; R^{3IV} represents a hydrogen atom or an amino group; R^{4IV} represents a hydrogen atom, a lower alkyl group or an amino group; R^{5IV} represents a hydrogen atom or a dimethylamino group; X^{IV-} represents an anion; and Y^{IV} represents a sulfur atom or oxygen atom,
a compound of the formula (V): 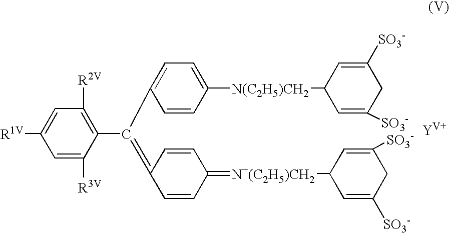
wherein R^{1V} represents a hydrogen atom or a hydroxyl group; R^{2V} represents a hydrogen atom or a sulfonic group; R^{3V} represents a hydrogen atom or a sulfonic group; and Y^{V+} represents an alkali metal cation,
and the group consisting of NK-2825, NK-1836, NK-1954, Oxazine750, cryptocyanine, NK-376, NK-382, NK-2711, NK-138, Oxazine720, LDS730, LD700, Nile Blue A, Brilliant Green, Iodide Green and Malachite Green.

8. A method as claimed in claim 1, wherein an agent for lysing erythrocytes in the first lysing agent is an aqueous solution having an osmotic pressure of 100 m Osm/kg or less and a pH of about 2.0 to 5.0.

9. A method as claimed in claim 1, wherein the first lysing agent comprises one or more surfactants selected from the following group:
a compound of the formula (VI): 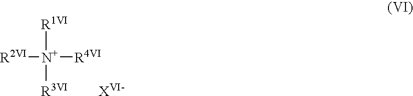
wherein, R^{1VI}, R^{2VI} and R^{3VI} respectively represent a hydrogen atom, a C₁₋₈ alkyl group or a C₆₋₈ aralkyl group; R^{4VI} represents a C₈₋₁₈ alkyl group, a C₈₋₁₈ alkenyl group or a C₆₋₁₈ aralkyl group; and X^{VI-} represents an anion,
a compound of the formula (VII): 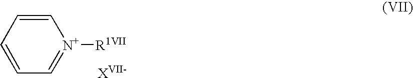
wherein R^{1VII} represents a C₈₋₁₈ alkyl group; and X^{VII-} represents an anion,
a compound of the formula (VIII): 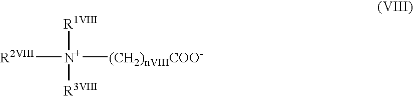
wherein R^{1VIII} and R^{2VIII} respectively represent a hydrogen atom, a C₁₋₈alkyl group or a C₆₋₈ aralkyl group; R^{3VIII} represents a C₈₋₁₈ alkyl group, a C₈₋₁₈ alkenyl group or a C₆₋₁₈ aralkyl group; and nVIII represents 1 or 2,
a compound of the formula (IX),
R^{1IX}—R^{2IX}—(CH₂CH₂O)_{nIX}—H (IX)
wherein R^{1IX} represents a C₉₋₂₅ alkyl group, a C₉₋₂₅ alkenyl group or a C₉₋₂₅ alkynyl group; R^{2IX} represents a group of formula 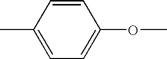
or —COO—; nIX represents from 10 to 40, and
the class consistig of MEGA-8, sucrose monocaprate, deoxy-BIGCHAP, n-octyl-β-D-thioglucoside, n-nonyl-β-D-thiomaltoside, n-heptyl-β-D-thioglucoside, n-octyl-β-D-glucoside, CHAPS and CHAPSO

10. A method as claimed in claim 9, wherein the concentration of the surfactant in the first lysing agent is 10 to 10,000 mg/L.

11. A method as claimed in claim 1, wherein the second lysing agent comprises;
(1) a nonionic surfactant of polyoxyethylenes for solidifying cytoplasms and cell membranes of the immature myeloid cells;
(2) a solubilizing agent for damaging the cell membrane of hematocytes to shrink them;
(3) an amino acid for solidifying the cytoplasm and cell membrane of immature myeloid cells; and
(4) a buffer which renders the solution adjusted to pH 5.0 to 9.0 and osmotic pressure to 150 to 600 m Osm/kg.

12. A method as claimed in claim 1, wherein the second fluorescent dye to produce a difference in intensity of fluorescence between the first group containing mature myeloid cells and lymphoid cells and the second group containing immature myeloid cells is one or more dyes selected from the following group:
a compound of the following formula (X): 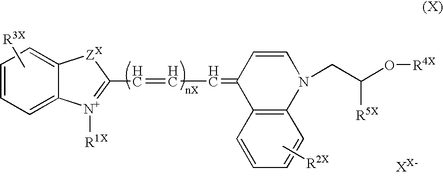
wherein R^{1X} represents a hydrogen atom or a lower alkyl group; R^{2X} and R^{3X} respectively represent a hydrogen atom, a lower alkyl group or a lower alkoxyl group; R^{4X} represents a hydrogen atom, an acyl group or a lower alkyl group; R^{5X} represents a hydrogen atom or a lower alkyl group optionally substituted; Z^{X} represents a sulfur atom, an oxygen atom or a carbon atom substituted with two lower alkyl groups; n_{X} represents 1 or 2; X^{X-} represents an anion;
ethidium bromide, propidium iodide, ethidium-acridine, heterodimer, ethidium azide, ethidium homodimer-1, ethidium homodimer-2, ethidium monoazide, TOTO-1, TO-PRO-1, TOTO-3, and TO-PRO-3.

13. A method as claimed in claim 1, wherein the scattered light for measuring is at least one selected from side scattered light, forward low-angle scattered light, and forward high-angle scattered light.

14. The method claim 1, wherein the mixture of the first sample and first lysing agent has a suitable state for staining leukocytes, erythroid cells and lipid particles.

15. The method claim 1, wherein the mixture of the second sample and second lysing agent has a suitable state for staining mature myeloid cells, lymphoid cells and immature myeloid cells.

16. An automatic analyzer for analyzing nucleated cells of bone marrow fluid comprising:
a dispenser for preparing a first and second samples from a sample of bone marrow fluid;
a first reaction chamber for preparing a first measuring sample, the first measuring sample being obtained by mixing the first sample with a first lysing agent to lyse erythrocytes in the first sample, and mixing the mixture with a first staining solution containing at least a first fluorescent dye for producing a different in each intensity of fluorescence among at least leukocytes, erythroid cells and lipid particles;
a second reaction chamber for preparing a second measuring sample, the second measuring sample being obtained by mixing the second sample with a second lysing agent to damage all the cells but immature myeloid cells, and mixing the mixture with a second staining solution containing at least a second of fluorescent dye for producing a different in each intensity of fluorescence between a group of mature myeloid cells and lymphoid cells and a group of immature myeloid cells;
a flow cytometer for measuring a scattered light and a fluorescence of the first measuring sample and the second measuring sample; and
a data processor for obtaining a ratio of myeloid cells and erythroid cells (M/E ratio) by classifying and counting leukocytes, erythroid cells and lipid particles with use of a difference in each intensity of the scattered light and the fluorescence of the first measuring sample, classifying and counting mature myeloid cells, lymphoid cells and immature myeloid cells with use of difference in each intensity of the scattered light and the fluorescence of the second measuring sample, calculating the number of myeloid cells from the counted mature myeloid cells and immature myeloid cells, and calculating the counted erythroid cells and myeloid cells.

17. An automatic analyzer as claimed in claim 16, wherein the dispenser comprises a sampling valve.

18. An automatic analyzer as claimed in claim 16, wherein the flow cytometer has a detector for forward scattered light, a detector for side scattered light and a detector for fluorescence.

19. An automatic analyzer as claimed in claim 18, wherein the detector for forward scattered light contains a low-angle forward scattered light or a high-angle forward scattered light.

20. An automatic analyzer as claimed in claim 16, wherein the data processor is a personal computer.
